(19) United States
(12) Patent Application Publication
(10) Pub. No.: US 2012/0129927 A1
(43) Pub. Date: May 24, 2012

(54) New Anthraquinone Derivatives

(75) Inventors: Alexander Pretsch, Grossmugl (AT); Peter Proksch, Julich (DE); Abdessamad Debbab, Dusseldorf (DE)

(73) Assignee: SEALIFE PHARMA GMBH, Tullin (AT)

(21) Appl. No.: 13/375,084

(22) PCT Filed: May 31, 2010
(86) PCT No.: PCT/AT10/00190

§ 371 Date: Feb. 7, 2012

(30) Foreign Application Priority Data
May 29, 2009 (AT) A 842/2009

(51) Int. Cl. A61K 31/222; C07C 49/755; A61K 31/122; C12P 15/00; A61P 31/14; C07C 67/26; A61P 35/00; A61P 31/04; A61P 31/12; A61P 31/10; C07C 69/157; C07C 67/10

(52) U.S. Cl. 514/510; 552/261; 552/212; 514/680; 435/127

(57) ABSTRACT

The invention relates to two novel substances, namely (1R,2S,3S,4R)-3-acetoxy-1,2,4,5-tetrahydroxy-7-methoxy-2-methyl-1,2,3,4-tetrahydroanthracene-9,10-dione (3-O-acetyl altersolanol M)

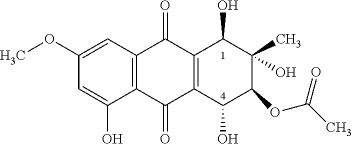

and 8-(4,5,6-trihydroxy-7-methyl-2-methoxy-9,10-dioxo-9H,10H-anthracen-1-yl)-(1S,2S,3R,4S)-1,2,3,4,5-pentahydroxy-7-methoxy-2-methyl-1,2,3,4-tetrahydro-9H,10H-anthracene-9,10-dione (the atropisomers alterporriol I and J)

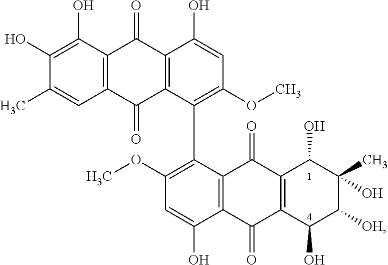

to their use as anti-infectives and as anti-cancer agents, as well as to methods of producing the same.

The present invention relates to new anthraquinone derivatives, a method for producing them and for using them as anti-infectives, especially against multiply drug-resistant pathogens, as well as anti-cancer agents.

### STATE OF THE ART

In the late 1960s and early 1970s, it was assumed, due to the highly successful use of anti-microbial drugs, that infectious diseases would not constitute any danger any more. This, however, proved to be a misconception, all the more so as, forty years later, microbes are constituting a greater threat than ever before. For this reason, there is an urgent need for new anti-microbial agents. These days, infectious diseases constitute the third most frequent cause of death in the US, and the second most frequent cause of death at a global level. Ineffective anti-microbial drugs are responsible for most of these cases, and the resistance of certain bacteria and fungi to these agents confronts our society with a serious problem. According to statistical data from the US, the majority of infections contracted in hospitals (the so called nosocomial infections) is caused by a small number of bacteria species, namely by Enterococcus faecium, Staphylococcus aureus, Klebsiella pneumoniae, Acinetobacter baumanii, Pseudomonas aeruginosa, and Enterobacter sp., which, based on their starting letters, are collectively referred to as “ESKAPE” pathogens (Boucher et al., “IDSA Report on Development Pipeline”, CID 2009:48, Infectious Disease Society of America, Jan 1, 2009). At the same time, this refers to the fact that these resistant pathogens escape the effect of anti-bacterial drugs, particularly as a resistance to antibiotics, at the molecular level, is nothing else than the ability acquired by a microorganism to resist the growth inhibiting or bactericidal action of an anti-microbial substance. This means that the substance becomes clinically ineffective. A “methicillin-resistant Staphylococcus aureus” (abbreviated as MRSA; this abbreviation being, however, also used in a more general sense for “multi-resistant Staphylococcus aureus”) means that the use of β-lactams is ineffective in the therapy of S. aureus, while glycopeptides often still have an effect. It is, thus, absolutely necessary to test the action new anti-infectives have on multi-resistant strains, as, even though they are effect against species and strains which are sensitive to them, this does not automatically mean that they are also effective against multi-resistant bacteria, just like an agent against Gram-positive bacteria is not automatically effective against Gram-negative bacteria and vice versa (see Boucher et al., supra, etc.)

Apart from strategies against bacteria and fungi, we currently also lack effective strategies against respiratory viruses. In most cases, it is just the symptoms which are cured, without fighting the virus itself. A solution for the future could consist mainly in combinations of active agents.

In the past, numerous substances for fighting the proliferation of microorganisms were found in endophytic fungi. In a number of cases, these substances have very good anti-bacterial, anti-fungal, and anti-viral activities and may be used for multiple applications (G. A. Strobel, Crit. Rev. Biotechnol. 22, 315-333 (2002)). Due to the fact that research in this field was carried out mainly at an academic level and did not directly aim at the development of new active agents, there are hardly any suitable drugs commercially available today. Especially screening against resistant microbes was neglected in the past, so that only a few substances against drug-resistant microbes have been tested. The situation is even worse in the case of respiratory viruses; so far, hardly any substances effective against respiratory viruses have been screened.

Already in 1969, A. Stoessl, Can. J. Chem. 47, 767 (1969) described the isolation of a new metabolic pigment from Alternaria solani, a mold fungus causing a disease called early blight in potatoes. This pigment was named altersolanol A, and its structure was identified to look as follows:

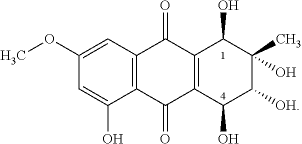

Thus, the chemical name of this anthraquinone is 7-methoxy-2-methyl-(1R,2S,3R,4S)-1,2,3,4-tetrahydro-1,2,3,4,5-pentahydroxy-anthracene-9,10-dione.

Subsequently, this pigment as well as several isomers and derivatives thereof were detected in further Alternaria species (such as Alternaria porri) and in some other fungal genera (see, for example, R. Suemitsu et al., Agric. Biol. Chem. 45(10), 2363-2364 (1981)). The majority of the isomers and derivatives corresponds to one of the following general formulae (1) and (2):

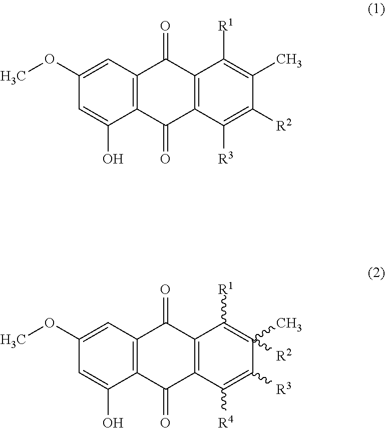

wherein R¹ to R⁴ may each represent H or OH, which, in the case of OH in formula (2), results in one chiral carbon atom, which may be in R- or S-configuration.

Subsequently, dimers, i.e. bisanthraquinones, were discovered as of metabolites, first in Alternaria porri, another representative of the genus Alternaria, which, amongst others, causes the purple blotch disease in onions, which is why these dimers were named alterporriols. They, for example, correspond to the following formula (3):

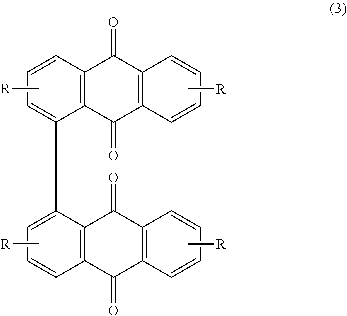

wherein the possible number of diverse substitution and hydration patterns of the aromatic rings is similar to that in “monomeric” altersolanols. Due to the restricted rotational freedom about the axis of the chemical bond between the anthraquinone cores, a high number of alterporriol derivatives exist in the form of two atropisomers.

There are reports on some compounds referred to as altersolanols and alterporriols concerning their effectiveness against certain microorganisms, while others have been described as not having any anti-infective effect. Aly et al., Phytochemistry 69, 1716-1725 (2009), for example, examined bioactive metabolites of endophytic fungi from the genus Ampelomyces as well as their anti-infective action. Among other things, they found out that altersolanol J, the alterporriols D and E (atropisomers) as well as the compound ampelanol, which is closely related to altersolanols, do not show any activity against bacteria and fungi, whereas altersolanol A proved to be the most efficient of the tested substances. Moreover, Okamura et al., Phytochemistry 42(1), 77-80 (1996), for example, do not disclose any effect of tetrahydroaltersolanol B against Gram-positive bacteria and the Gram-negative species Pseudomonas aeruginosa. Yagi et al., Phytochemistry 34(4), 1005-1009 (1993) report on the anti-microbial activity of the altersolanols A, B, C, and E, whereas the altersolanols D, E and F proved to be completely ineffective in the same test. US application No. 2007/258913, on the other hand, discloses the atropisomeric altersolanol D and E as compounds which are suitable for preventing the formation of a biofilm in the oral cavity, only in a very general way, though, and without listing any specific data concerning their effectiveness.

Thus, it is impossible to predict whether a specific altersolanol or alterporriol isomer or derivative will show an anti-infective effect or not, and still less against which genera or species of microorganisms.

For some years, similar trends as in the field of anti-infectives have become apparent in the field of anti-cancer agents, that is a continuous increase of resistances against existing therapies. In this case, the development of resistances is mainly due to the fact that an overexpression of integral membrane transporters, such as P-gp, leas to an efflux of active agents from the cancerous cell, so that they can no longer become effective. By this time, such multi-resistant (“multiply drug resistant”, MDR) cells have developed resistances against numerous structurally and mechanistically diverse chemotherapeutics (see, for example, E. L. Cooper, Evid. Based Complement. Alternat. Med. 1, 215-217 (2004); M. D'Incalci et al., J. Chemother. 16 (Suppl. 4), 86-89 (2004); Z. Shi et al., Cancer Res. 67, 11012-11020 (2007)). Thus, apart from developing new anti-infective substances against pathogens, the discovery and application of new active agents against cancer has become desirable, in order to suppress the development of resistances.

For several decades, anthraquinones have, amongst others, been used as effective chemotherapeutics in cancer therapy. The structurally similar active agents doxorubicin (which is exemplarily illustrated below), daunorubicin, idarubicin, and epirubicin, which may be used against various types of cancer, are well-known drugs from this group.

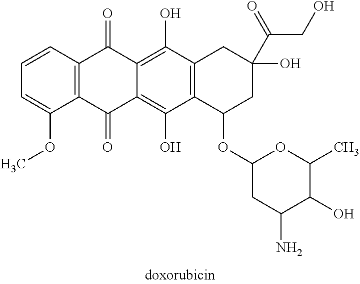

Apart from their desired therapeutic effect, such active agents also show the negative effect of forming of superoxide radicals. Due to the radical-stabilizing effect of the quinone group in the molecule, anthraquinones frequently lead to such a formation of radicals, which, when such molecules are used for therapeutic purposes, may result in cardiotoxicity, amongst others. Both the therapeutic and the radical-stabilizing effect of the quinone group are significantly influenced by substituents. See, for example, the investigations carried out with mouse lymphoma cells by Debbab et al., J. Nat. Prod. 72(4), 626-631 (2009).

It is assumed that the mechanism of action of the cytotoxic effect of anthraquinones is mainly due to an intercalation of the molecule into the DNA's double helix, which prevents both the replication and the transcription of DNA into RNA from taking place properly. In addition to this effect, anthraquinones are assumed to inhibit type II topo-isomerase which, amongst others, is responsible for the super-spiralization of DNA and to sometimes induce apoptosis. Whether an anthraquinone or another similarly acting, i.e. intercalating, molecule can be successfully introduced into the double helix significantly depends from the types and positions of substituents, as mentioned above. Current investigations using different anthraquinone derivatives (see, for example, J. Zhang et al., Mar. Drugs 8, 1469-1481 (2010)) have shown that very slight differences between the ring substituents are decisive for the molecule's cytotoxic activity.

Against this backdrop, the aim of the present invention consisted in the identification, isolation, and production of new substances to be used as anti-infective agents or anti-cancer agents in pharmaceutical compositions, particularly of compounds showing an activity against multi-resistant (“multiply drug resistant”, MDR) pathogens and cells.

In the course of their research work, the inventors were able to identify several substances—some of them having so far unpublished structures, i.e. new chemical compounds—which show good activities against microorganisms and respiratory viruses, especially against MDR pathogens, but also against cancer cells. While the parallel, simultaneously filed Austrian Patent Application with the Application No. A 843/09 pertains to several compounds of a known structure, the effectiveness of which, however, has been unknown so far, the present invention relates to providing two novel compounds, one altersolanol and one alterporriol derivative.

### DISCLOSURE OF THE INVENTION

More specifically, the inventors have produced, isolated and characterized the following novel compounds:

(1R,2S,3S,4R)-3-acetoxy-1,2,4,5-tetrahydroxy-7-methoxy-2-methyl-1,2,3,4-tetrahydroanthracene-9,10-dione according to formula (4), which was originally named “altersolanol M” by the inventors, but has now been renamed “3-O-acetyl altersolanol M” (because, according to internationally common practice, the characteristic letter is used to refer to the non-acetylated forms):

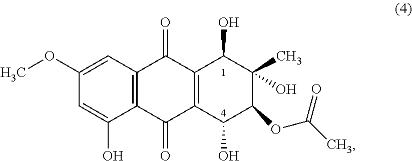

and 8-(4,5,6-trihydroxy-7-methyl-2-methoxy-9,10-dioxo-9H,10H-anthracen-1-yl)-(1S,2S,3R,4S)-1,2,3,4,5-pentahydroxy-7-methoxy-2-methyl-1,2,3,4-tetrahydro-9H,10H-anthracene-9,10-dione according to formula (5), existing in the form of two atropisomers which have been named alterporriol I and J by the inventors:

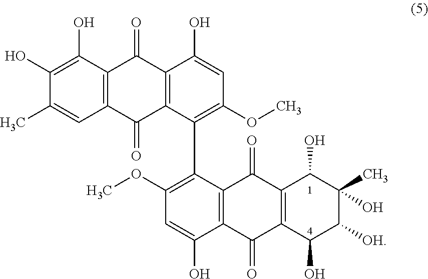

As both compounds (4) and (5) show an excellent anti-microbial, anti-viral and also, especially in the case of compound (4), an excellent anti-cancer effect in screening experiments, a second aspect of the present invention consists in the use of these novel compounds as anti-infectives, preferably against Gram-positive and Gram-negative bacteria, fungi, and respiratory viruses, particularly as anti-infectives against multiply drug resistant (MDR) pathogens, as well as in their use as anti-cancer agents.

The two compounds are preferably used against multiply drug resistant strains of meth icillin-resistant Staphylococcus aureus (M RSA), Staphylococcus epidermis, Streptococcus pneumoniae, Enterococcus faecalis or faecium, Escherichia coli, Klebsiella sp., Pseudomonas aeruginosa, Aspergillus sp., as well as against respiratory viruses from the group of human rhinoviruses and respiratory syncytial viruses, which will be proved in detail by exemplary embodiments below.

Further acetylated derivatives of altersolanol M, i.e. both isomers of 3-O-acetyl altersolanol M having the acetyl group located in another position such as 4-O-acetyl altersolanol M and 5-O-acetyl altersolanol M, and derivatives having several acetyl groups such as 3,4-di-O-acetyl- and 3,5-di-O-acetyl altersolanol M, were also tested in pre-experiments for their anti-infective or cytotoxic action and, partly, also yielded excellent results, so that further research work of the inventors will focus on the production and investigation of these compounds.

The present invention also relates to a method for producing compounds according to the formulae (4) and (5), said method consisting in fermenting a microorganism producing the compound or one of its precursors under growth conditions and obtaining the respective compound from the culture, optionally after disrupting the cells of the microorganism in order to increase the yield. Such a fermentation method preferably uses a pure strain of Stemphylium globuliferum, as the inventors could obtain the highest yields using this species. Alternatively, any other microorganism capable of producing the new compounds or their precursors, e.g. Alternaria sp., may be used for fermentation. If the fermentation yields precursors, said precursors may be converted into the desired new compounds of formulae (4) and (5) by any methods known to those skilled in the art of organic synthesis, optionally carrying out some steps of the method under the influence of enzymatic catalysis, in order to obtain higher enantioselectivities.

For example, it is possible to cultivate Alternaria sp. in order to obtain altersolanol A, or another member of the altersolanol family, which may be converted into 3-O-acetyl altersolanol M or a stereoisomer thereof by acetylating the OH group on C3 (optionally protecting the other OH groups using conventional protecting groups), from the fermentation broth. Another synthesis pathway consists in cleaving the OH group from C3 or C4, together with the adjacent hydrogen atom, i.e. by dehydration, in order to obtain a double bond between C3 and C4 and two prochiral centers in these positions. Subsequently, it is possible to enzymatically rehydrate this double bond, which will yield the desired altersolanol M if the stereospecifity of the enzyme (such as hydratase, peroxygenase) is suitably selected. The alterporriols I and J may, for example, be obtained by chemically (again, for example, enzymatically) linking the corresponding anthraquinone substituent to position 8 of altersolanol M, which may optionally have been derivatized before, whereafter the acetyl group is hydrolytically cleaved from the OH group in position 3.

Suitable enzymes for the synthesis steps for converting the precursors of the desired compounds may, in some cases, also be isolated from the microorganism used for fermentation or from another microorganism. In the latter case, it may be useful when, for example, a microorganism produces the desired product, but only in minor amounts or in a contaminated form so that, from an economic point of view, it is better to obtain a precursor of the product by cultivating another strain (or even another species or genus) and to use enzymatic synthesis in order to convert said precursor into the target compound.

The invention finally relates to methods for producing 3-O-acetyl altersolanol M, i.e. compound (4) of the present invention, by chemical synthesis according to any one of the following synthesis pathways A to C.

A) By syn-dihydroxylation

Starting from (1R,2R)-1,2,5-trihydroxy-7-methoxy-2-methyl-1,2-dihydroanthracene-9,10-dione (6), which may be obtained in a way known from literature (Krohn et al., Liebigs Ann. Chem. 1988, 1033-1041), 3-O-acetyl altersolanol M may be obtained by the following steps:

a) syn-dihydroxylation of (6) at C3 and C4 (according to Houben-Weyl, Handbuch der organischen Chemie, vol. E21d, “Stereoselektive Synthese”, pages 4581-4587) to obtain (1R,2S,3R,4R)-3-acetoxy-1,2,4,5-tetrahydroxy-7-methoxy-2-methyl-1,2,3,4-tetrahydroanthracene-9,10-dione (7):

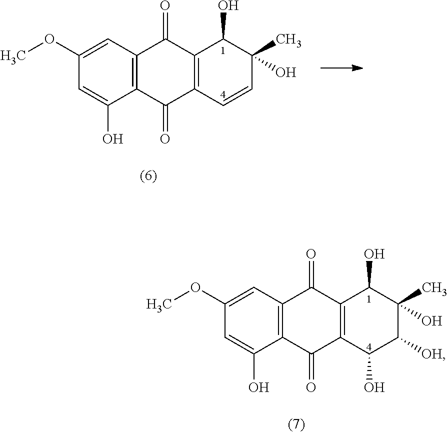

b) conversion of (7) (according to Y. Gao and K. B. Sharpless, J. Am. Chem. Soc. 110, 7538-7539 (1988)) into the cyclic sulfate (8):

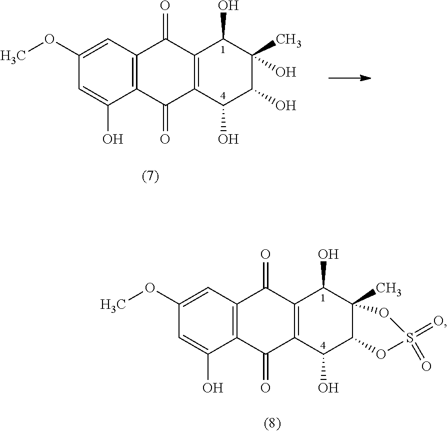

c) acidolysis of the cyclic sulfate (8) (according to H.-S. Buyn et al., Tetrahedron 56, 7051-7091 (2000)) using acetic acid to obtain 3-O-acetyl altersolanol M according to formula (4):

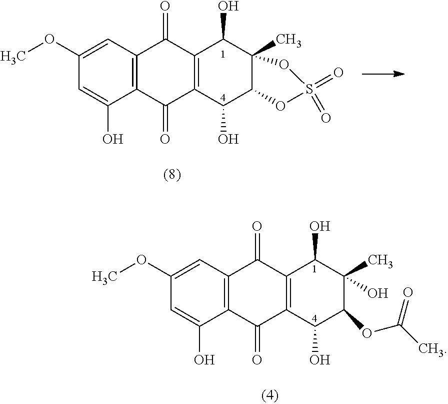

In contrast to the method for producing (amongst others) altersolanol A and altersolanol M described by Krohn et al. (supra), the method of the invention does not oxidize the double bond of compound (6) between C3 and C4 using meta-chloroperbenzoic acid in order to obtain the corresponding epoxide, as this step inevitably yields a diastereomeric mixture of cis- and trans-epoxy alcohols, which significantly reduces the yield in the desired isomer. By applying syn-dihydroxylation in step a) according to the invention, it is possible to selectively obtain the desired isomer, which may then be converted into the target compound 3-O-acetyl altersolanol M by a simple acidolysis using acetic acid.

### B) By Diels-Alder Addition and Epoxide Formation

Starting from protected 5-hydroxy-7-methoxy-1,4-naphthoquinone (9), which may be obtained in a way known from literature (Krohn et al., Liebigs Ann. Chem. 1988, 1033-1041), 3-O-acetyl altersolanol M may be obtained by the following steps:

a) Diels-Alder addition between naphthoquinone (9) and methylbutadiene (10), wherein the R independently are identical or different hydroxyl protecting groups, in order to obtain the tetrahydroanthraquinone (11):

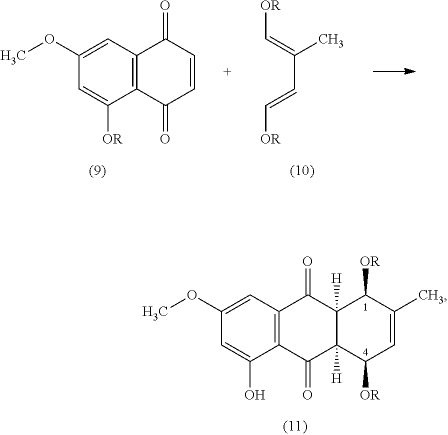

b) oxidation of the double bond between C2 and C3 using meta-chloroperbenzoic acid (according to Krohn et al. (supra), carrying out a subsequent or precedent Mitsunobu reaction in order to invert the oxygen functionality at C4, in order to obtain the epoxide (12):

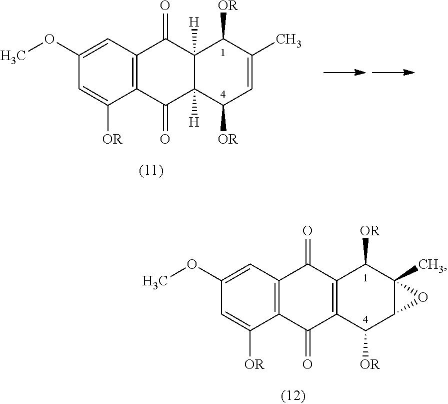

c) acidolysis of the epoxide (12) using acetic acid, subsequently or simultaneously cleaving the protecting groups R, in order to obtain 3-O-acetyl altersolanol M.

In contrast to the Diels-Alder reaction described by Krohn et al., the reaction of the present invention already uses a butadiene (10) having two oxygen functionalities, i.e. protected hydroxyl groups, which are diastereoselectively integrated into the anthraquinone (11) at C1 and C4 at the same time, said anthraquinone (11) being convertible into the epoxide (12) by a single oxidation step, as the substituents at C1 and C2 have a directing effect on the oxidation. Thus, the Mitsunobu reaction for inverting the oxygen functionalities at C4 is preferably carried out after the oxidation. The acidolysis following the latter is also carried out stereoselectively, as the nucleophilic attack of the acetate at C3 from the side opposite the epoxide's oxygen is clearly favored.

### C) By Diels-Alder Addition and Syn-Dihydroxylation

Again starting from the protected 5-hydroxy-7-methoxy-1,4-naphthoquinone (9) 3-O-acetyl altersolanol M may also be obtained by the following steps:

a) Diels-Alder addition between naphthoquinone (9) and methylbutadiene (10), wherein the R independently are identical or different hydroxyl protecting groups, to obtain the tetrahydroanthraquinone (11), as described above for method B):

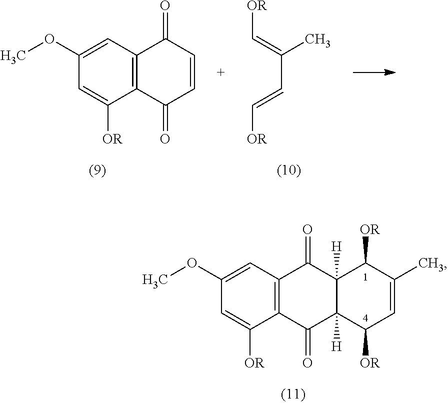

b) syn-dihydroxylating the tetrahydroanthraquinone (11) (according to Houben-Weyl, supra), carrying out a subsequent or precedent Mitsunobu reaction in order to invert the oxygen functionality at C4, in order to obtain the tetrahydroanthraquinone (13):

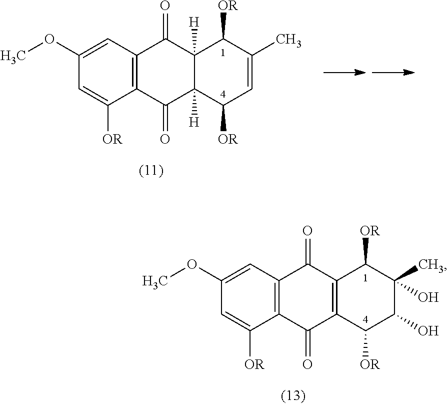

c) converting (13) into the cyclic sulfate (14) (according to Y. Gao and K. B. Sharpless, supra):

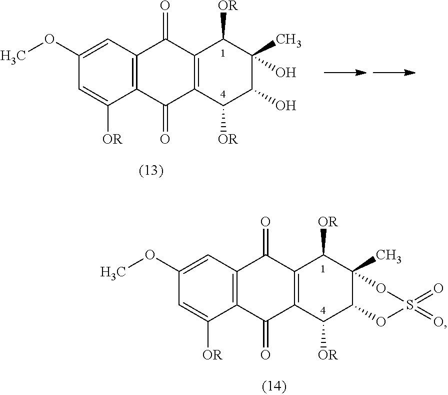

d) acidolysis of the cyclic sulfate (14) using acetic acid (according to H.-S. Buyn et al., supra), subsequently or simultaneously cleaving the protecting groups R, in order to obtain 3-O-acetyl altersolanol M.

In contrast to method B), the tetrahydroanthraquinone (11) is not oxidized to obtain an epoxide, but is diastereoselectively oxidized in order to obtain the dihydroxy derivative (13), which, as described above for method A), may easily be converted into a cyclic sulfate and subsequently into 3-O-acetyl altersolanol M.

Instead of cyclic sulfates, the above diols may also be converted into other cyclic esters such as cyclic ortho-esters, which are also included in the scope of the method of the present invention.

Once the inventors will have completed the ongoing experiments for optimizing the yields, chemical synthesis will certainly become the method of choice for the production of 3-O-acetyl altersolanol M, but also of alterporriol I and J, due to the higher purities and better isolatabilities of the thus obtained products, as compared to fermentation methods. A synthesis strategy for the production of the compounds of formula (5) is currently being developed by the inventors.

Currently, the respective compound (4) or (5) of the present invention is still obtained preferably by extraction and subsequent isolation from a crude extract of a culture, for example by fractional crystallization or chromatographic procedures, more preferably by preparative HPLC, which has resulted in the highest yields and highest purities at the same time. Of course, other methods of isolation are also contemplated, particularly in larger-scale experiments, e.g. by direct fractional crystallization of the crude extracts or by absorption procedures. However, one skilled in the art will be able to determine the best suitable isolation procedures for the respective cultivation (and/or synthesis or partial synthesis) steps without undue experimentation.

### EXAMPLES

Below, the invention will be described in further detail referring to specific exemplary embodiments, which, however, are not intended to limit the invention's scope in any way.

The new compounds of the invention were obtained by cultivating Stemphylium globuliferum and subsequent extraction.

### Isolating the Microorganism

Fresh, healthy stems of Mentha pulegium (pennyroyal) were used for isolating the endophytic fungus. The surface of the stems was sterilized using 70% ethanol for 1 min and then rinsed with sterile water in order to remove the alcohol. In order to distinguish any remaining epiphytic fungi from endophytic fungi, an imprint of the stem surface was obtained on organic malt extract agar. Small tissue samples from the interior were aseptically sectioned and pressed onto agar plates containing an antibiotic in order to suppress bacterial growth. The composition of the isolating medium was as follows: 15 g/1 malt extract, 15 g/l agar, and 0.2 g/l chloroamphenicol in distilled water, pH 7.4-7.8. A pure fungus strain was obtained from the cultures by repeated inoculation on malt extract agar plates.

The fungus culture was identified as Stemphylium globuliferum according to a molecular biological protocol by means of DNA amplification and sequencing of the IST region. The sequence data was deposited with GenBank under access number EU859960.

### Cultivation

For cultivating Stemphylium globuliferum, 1 liter Erlenmeyer flasks, each containing 100 g rice and 100 ml distilled water, were autoclaved in order to contain a swollen, solid rice medium. A small part of the above isolating medium, containing the pure fungus strain, was applied to the solid rice medium under sterile conditions, and the fungus was cultivated at room temperature (20° C.) for 40 days.

### Extraction and Isolation

After 40 days, the culture was extracted twice with 300 ml ethyl acetate (EtOAc). The EtOAc extraction was extracted by shaking out using water. The aqueous phase was evaporated to dryness, and the residue was subjected to column chromatography on a LH-20 column using 100% methanol (MeOH) as eluant, while being detected by means of thin layer chromatography (TLC) on silica F245 (Merck, Darmstadt, Germany) using EtOAc/MeOH/H₂O (77:13:10) as eluant. The fractions containing the desired compounds were combined and subjected to semi-preparative HPLC (Merck, Hitachi L-7100) using a Eurosphere 100-10 C18 column (300×8 mm, L×i.d.) and a linear water-methanol gradient. This way, the novel compounds of the invention, i.e. (1R,2S,3S,4R)-3-acetoxy-1,2,4,5-tetrahydroxy-7-methoxy-2-methyl-1,2,3,4-tetrahydroanthracene-9,10-dione (3-O-acetyl altersolanol M) and 8-(4,5,6-trihydroxy-7-methyl-2-methoxy-9,10-dioxo-9H,10H-anthracen-1-yl)-(1S,2S,3R,4S)-1,2,3,4,5-pentahydroxy-7-methoxy-2-methyl-1,2,3,4-tetrahydro-9H,10H-anthracene-9,10-dione (atropisomers alterporriol I and J), were obtained in their pure forms.

### Analytics

### Alterporriol I and J

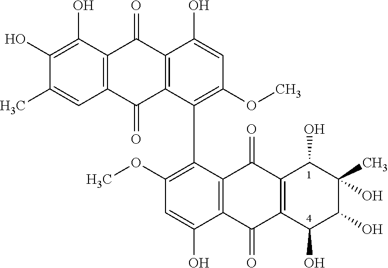

Based on the HRESI MS spectrum of alterporriol I and J, it becomes apparent that the molecular ion is located at m/z 635,1406 [M+1]⁺, leading to the empirical formula C32H₂₆O₁₄ and 20% unsaturations. The data of the ¹H- and ¹³C-NMR spectra as well as of the ESI/MS spectrum (m/z 634.9 [M+H]⁺) are similar to those of the known related anthraquinones altersolanol A and 6-O-methylalaternin. The type of the bond between the two anthraquinone units becomes apparent from the interpretation of the COSY- and HMBC spectra and from the comparison to NMR data of the compounds altersolanol A and 6-O-methylalaternin. The ¹H-NMR spectrum listed in Table 1 below shows signals of two tertiary methyl groups at δ 1.110 (CH₃-7′) and 1.114 (CH₃-7′*), two aromatic methyl groups at δ 2.17 (CH₃-2) and δ 2.16 (CH₃-2*), and four methoxy groups, which partially overlap and resonate at δ 3.68, 3.70, and 3.71 (derived from the integral). In addition, overlapping singlets at δ 4.04 (H-8′/H-8′*), overlapping doublets at δ 4.45 (H-5′/H-5′*), and overlapping doublets at δ 3.54 (H-6′/H-6′*) can be observed. The aromatic areas shows five proton resonances at δ 7.40, 6.94, 6.93, 6.91, and 6.90. Moreover, no meta-coupling protons are apparent in the ¹H-NMR of alterporriol. The COSY spectrum, however, shows clear long range correlations between CH₃-2 and H-1, proving that the 7-O-methylalaternin moiety is bound to the altersolanol A moiety either at C-6 or at C-8. The interpretation of the HMBC spectrum, also listed in Table 1, shows that the aromatic proton (H-6/H-6*) observed at δ 6.93/δ 6.94 strongly correlates with two oxygen-bearing carbon atoms at δ 165.1 and 165.6 (C-5 and C-7). The aromatic proton (H-3′/H-3′*) resonating at δ 6.90/6 6.91 correlates with two oxygen-bearing carbon atoms at δ 164.2 and 164.3 (C-2′ and C-4′), which indicates C-8 as joint between the two monomers.

Moreover, the interpretation of the HMBC spectrum in Table 1 shows that H-1 correlates with C-9 (δ 180.4), C-4a (δ 129.0), and C-3 (δ 150.9), while H-6/6* correlates with C-8 (δ 123.0) and C-10a (δ 109.3). Apart from that, it was found that H-3′ strongly correlates with the carbon atoms C-2′, C-4′ (δ 164.2, 164.3), C-1′ (δ 123.1), C-4′ a (δ 109.7), and the keto group at C-10′ (δ 188.8), the latter correlation being due to W-coupling.

In order to determine the relative configuration of alterporriols I and J in the aliphatic ring, a ROESY measurement was conducted. In this connection, the signal of CH₃-7′ shows a strong correlation with H-6′, H-8′, OH-6′ and OH-8′, indicating that CH₃-7′ is equatorial. Moreover, proton H-8′ correlates with proton H-6′, the latter being present in a diaxial relationship to H-5′ (which may be derived from the coupling constants). This way, the compounds alterporriol I and alterporriol J were identified as new natural products.

TABLE 1
¹H-NMR and ¹³C-NMR data of alterporriol I and J

| Atoms | ¹H | ¹³C | HMBC |
|---|---|---|---|
| 1 | 7.30 (s) | 122.5 | 3, 9, 2-Me, 4a |
| 2 | | 130.8 | |
| 3 | | 150.9 | |
| 4 | | 160.1 | |
| 4a | | 129.0 | |
| 5 | | 165.6* | |
| 6 | 6.93 (s)/6.94 (s) | 103.8 | 5, 7, 8, 10a |
| 7 | | 165.1* | |
| 8 | | 123.0 | |
| 8a | | 130.82 | |
| 9 | | 180.45 | |
| 9a | | 128.9 | |
| 10 | | 190.3 | |
| 10a | | 109.3 | |
| 1′ | | 123.1 | |
| 2′ | | 164.2# | |
| 3′ | 6.90 (s)/6.91 (s) | 103.8 | 1′, 2′, 4′, 4′a, 10′ |
| 4′ | | 164.3# | |
| 4′a | | 109.7 | |
| 5′ | 4.45 (d: 7.25) | 68.5 | 6′, 8′a, 10′a |
| 6′ | 3.54 (d: 7.25) | 73.7 | 5′, 8′ |
| 7′ | | 72.8 | |
| 8′ | 4.04 (s) | 68.3 | 6′, 7′, 8′a, 9′, 10′a, 2-Me |
| 8′a | | 142.52 | |
| 9′ | | 183.9 | |
| 9′a | | 123.5 | |
| 10′ | | 188.8 | |
| 10′a | | 143.5 | |
| 2-Me | 2.164/2.175 | 16.4/16.1 | 1, 2, 3, 4 |
| 7′-Me | 1.110/1.114 | 22.24 | 6′, 7′, 8′ |
| 7-OMe | 3.68/3.70 | 56.9/56.8 | 7 |
| 2′-OMe | 3.71 | 56.7 | 2′ |
| OH (4 & 4′) | 13.05 & 13.66 | | |
| OH | 4.84 | | |
| | 5.00 | | |
| | 5.46 | | |

### 3-O-acetyl Altersolanol M

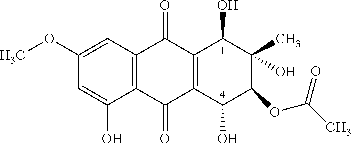

3-O-acetyl altersolanol M was isolated in the form of red-brown crystals. The HRESI-MS spectrum shows a main peak at m/z 379 ([M+H]⁺), which corresponds to 42 mass units (i.e. one acetyl group) more than in altersolanol A, resulting in the empirical formula C₁₈H₁₈O₉. The ¹H-NMR spectrum contains three exchangeable alcoholic hydroxyl groups, two doublets at δ 5.34 and 5.98, and one singlet at 4.86, which may, respectively, be assigned to 4-OH, 1-OH, and 2-OH. Moreover, two singlets at δ 1.13 and 2.11 (derived from the integral) can be observed, corresponding to two methyl groups. The meta-coupling protons resonate at δ 6.85 (H-6) and δ 7.04 (H-8), while the aromatic methoxy group is located at δ 3.91. In the COSY spectrum the high-field doublet (1-OH) correlates with the aliphatic low-field proton H-1; moreover, the proton H-4 appears at δ 4.68, correlating with the hydroxyl group (4-OH) on the carbon atom C4 and with the proton H-3. The overall aliphatic spin system exerts pressure on H-1, H-3, and H-4, which, together with the corresponding hydroxyl functionalities, clearly shows that the acetyl group is present in position C3. Moreover, the correlations (with 1, 2, 9, 9a, 2-Me) attributable to the protons, i.e. H-1, H-3, and H4, in the HMBC spectrum and the correlation of the aromatic protons fully support the above structure of 3-O-acetyl altersolanol M. The relative stereochemistry of 3-O-acetyl altersolanol M was derived from the coupling constants observed in the ¹H-NMR spectrum and from the correlations detected in the ROESY spectrum. The high value of J₁₋₂ (7.52 Hz) shows that H-4 and H-3 are present in a diaxial relationship, which is confirmed by the ROESY spectrum. In addition, the methyl group shows correlations with H-1,1-OH, 2-OH, and H-3 at δ 1.13, which may be explained by its equatorial position. Finally, proton H-1 shows a clear correlation with proton H-1, indicating the relative configuration of the aliphatic ring.

TABLE 2
¹H-NMR and ¹³C-NMR data of 3-O-acetyl altersolanol M

| Atoms | ¹H | ¹³C | HMBC |
|---|---|---|---|
| 1 | 4.37 d (5.94) | 68.61 | 1, 2, 9a, 9, 2-Me |
| 2 | | 72.22 | |
| 3 | 5.25 d (7.62) | 76.75 | 4, 11 |
| 4 | 4.68 dd (6.23. 7.59) | 65.92 | 4a |
| 4a | | 143.84 | |
| 5 | | 163.20 | |
| 6 | 6.85 d (2.52) | 106.07 | 7, 8, 4a, 5 |
| 7 | | 165.55 | |
| 8 | 7.04 d (2.49) | 106.80 | 4a, 6, 7, 9 |
| 8a | | 133.24 | |
| 9 | | 183.36 | |
| 9a | | 141.99 | |
| 10 | | 187.78 | |
| 10a | | 109.55 | |
| 11 | | 170.20 | |
| 2-Me | 1.13 | 21.76 | 1 |
| 11-Me | 2.11 | 20.91 | 11 |
| 6-OMe | 3.91 | 56.32 | 7 |
| 1-OH | 5.98 d (5.97) | | |
| 2-OH | 4.86 s | | 1 |
| 4-OH | 5.34 d (6.18) | | |

### Determination of Activity—Antimicrobial/Antifungal Action

The activities of the novel compounds were tested in two different screening systems. The antibacterial and antifungal activities were examined by means of an MIC test, MIC standing for “minimal inhibitory concentration” and referring to the lowest concentration of a substance at which no proliferation of microorganisms can be observed with the naked eye. The MIC is determined by means of a so called titration method in which the substance is diluted and, subsequently, the pathogen is added to it.

Usually, this method is applied in order to determine the concentration of an antibiotic which only just inhibits the growth of a bacterial strain. The MIC is indicated in micrograms per milliliter (μg/ml), and the dilutions are conventionally carried out in log 2 steps. Herein, a starting concentration of 250 μg/ml was diluted several times, to the double volume in each case, resulting in test concentrations of 250 μg/ml, 125 μg/ml, 62.5 μg/ml, 31.2 μg/ml, 15.6 μg/ml, 7.8 μg/ml, etc. Thus, lower values indicate a better activity as an anti-infective.

The tests were carried out according to the standards of EUCAST (European Committee for Antimicrobial Susceptibility Testing) and according to the AFST-protocol (“Antifungal Susceptibility Testing” protocol) of the European Society of Clinical Microbiology and Infectious Diseases (ESCMID).

The screening system for viruses is an infection system which involves the infection of host cells in vitro, the test substance being added before or after the infection in order to determine its activity. All these tests were carried out according to the internal standard protocol for drug screenings of SeaLife Pharma, using analogous dilution series as in the antibacterial/antifungal test above.

In the following Table 3, the test results indicating the anti-infective action of 3-O-acetyl altersolanol M, alterporriol I and J as well as of some known and structurally similar comparative substances against some multi-resistant bacteria and fungi (all of which were kindly placed at our disposal by Prof Georgopulos from the Medical University of Vienna). The data constituting averages of the values obtained in multiple test runs.

It becomes clear that the new compounds show excellent activities against four bacterial species, i.e. against Enterococcus faecalis or faecium, methicillin-resistant Staphylococcus aureus, Streptococcus pneumoniae, and Staphylococcus epidermis, and is, moreover, also effective against further pathogens, 3-O-acetyl altersolanol M showing a broader range of activity than alterporriol I and J in this series of tests.

TABLE 3
MHK values in multi-resistant bacteria and fungi

| Substances | EC | KL | PS | EK | MRSA | STR | SE | ASP |
|---|---|---|---|---|---|---|---|---|
| Example 1: 3-O-acetyl altersolanol M | 125 | 125 | 125 | 7.8 | 7.8 | 7.8 | 7.8 | 62.5 |
| Example 2: alterporriol I + J | | | | 7.8 | 7.8 | 7.8 | 7.8 | 125 |
| Bostrycin | | | | | 125 | 62.5 | 125 | |
| Altenusin | 62.5 | | | 62.5 | 31.2 | 31.2 | 31.2 | |
| Alterporriol A + B | | | | 15.6 | 15.6 | 7.8 | 15.6 | 62.5 |
| Altersolanol A | 62.5 | | | 15.6 | 7.8 | 7.8 | 7.8 | 7.8 |
| Altersolanol J | | 125 | | 31.2 | 62.5 | 15.6 | 62.5 | 125 |
| Ampelanol | | | | | | | | |
| Macrosporin | 62.5 | | | | 62.5 | | 62.5 | |
| Macrosporin sulfate | | | | | | 31.2 | | |
| Indole carbaldehyde (carboxaldehyde) | | | 125 | | | 62.5 | | |

| | |
|---|---|
| EC | E. coli |
| KL | Klebsiella sp. |
| PS | Pseudomonas aeruginosa |
| EK | Enterococcus faecalis or faecium |
| MRSA | methicillin-resistant Staphylococcus aureus |
| STR | Streptococcus pneumoniae |
| SE | Staphylococcus epidermis |
| AB | Acinetobacter baumanii |
| ASP | Aspergillus fumigatus or faecalis |

As a result of testing the activity of the new compounds of the invention against three different species of respiratory viruses, i.e. against a human rhinovirus (hrv), a respiratory syncytial virus (rsv), and paraflu, obtained from the ATCC, the inventors found out that, already at a concentration of 0.1 μg/ml, both (or all three) compounds provided the cells with 100% protection. Pre-tests using influenza and adenoviruses have also already yielded the first promising results.

### Determination of Activity—Cytotoxic Action

Alterporriol I and J and particularly 3-O-acetyl altersolanol M were tested for their anti-cancer action in several series of tests. The test series included both two-dimensional standard tests and a newly developed three-dimensional tumor model.

### a) Alamar Blue Assay Using Melanoma Cells

In this test, the dye resazurin is used as an indicator for measuring the cytotoxicity of substances. An aqueous, blue dyed resazurin solution is used, said solution being gradually reduced to resorufin by normally functioning cells (consuming NADH), resorufin being hot pink in color and fluorescent. Depending on the degree of their toxicity, cytotoxic agents decelerate or stop this reduction.

In the course of this experiment, melanoma cells were treated with several concentrations of 3-O-acetyl altersolanol M, i.e. with 1, 10, and 50 μg/ml, with altersolanol K and alterporriol F as comparative substances, as well as with alterporriol I+J, i.e. compound (5) of the present invention, at respective concentrations of 10 μg/ml, and incubated for 48 h, whereafter the samples' fluorescence was determined spectrophotometrically at 570/600 nm, the fluorescence being proportional to the number of surviving cells in the samples.

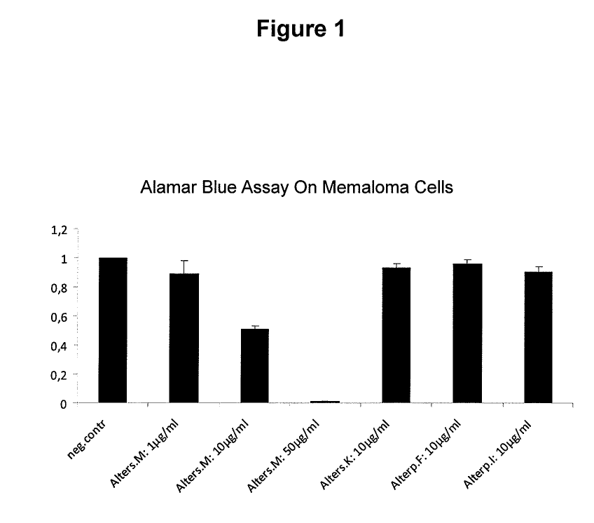

The measurement results were related to the fluorescence of a blank, to which no cytotoxic agent had been added, and are shown in a graph in FIG. 1. From left to right, the figure shows the results for the blank control (“neg. contr”), increasing concentrations of 3-O-acetyl altersolanol M (1, 10, and 50 μg/ml), as well as altersolanol K, alterporriol F, and Alterporriol I+J.

It can clearly be seen that, already at a concentration of only 1 μg/ml, 3-O-acetyl altersolanol M inhibited the number of surviving cells by more than 10%. These values were only achieved at a concentration of 10 μg/ml by altersolanol K, alterporriol F, and alterporriol alterporriol I/J of the invention yielding slightly better results than the two comparative examples. At this concentration of 10 μg/ml, 3-O-acetyl altersolanol M already achieved a reduction of the number of melanoma cells by about 50%, and at 50 μg/ml, it was hardly possible to detect any surviving cells. This means that in this test both compounds of the present invention showed cytotoxic activities, 3-O-acetyl altersolanol M being at least 10 times more effective, however.

Comparable results (not indicated herein) were also obtained in an Electrical CellSubstrate Impedance Sensing Technology- (ECIS-) test, 3-O-acetyl altersolanol M again achieving clearly better results than alterporriol I/J.

### b) Three-Dimensional Tumor Test

In addition to the above two-dimensional test, the cytotoxic action of 3-O-acetyl altersolanol M was also examined in an innovative three-dimensional test system. In this test, small tumors are grown in a matrix gel, in which the activity of an anti-cancer agent may be directly tested by adding said agent at different concentrations.

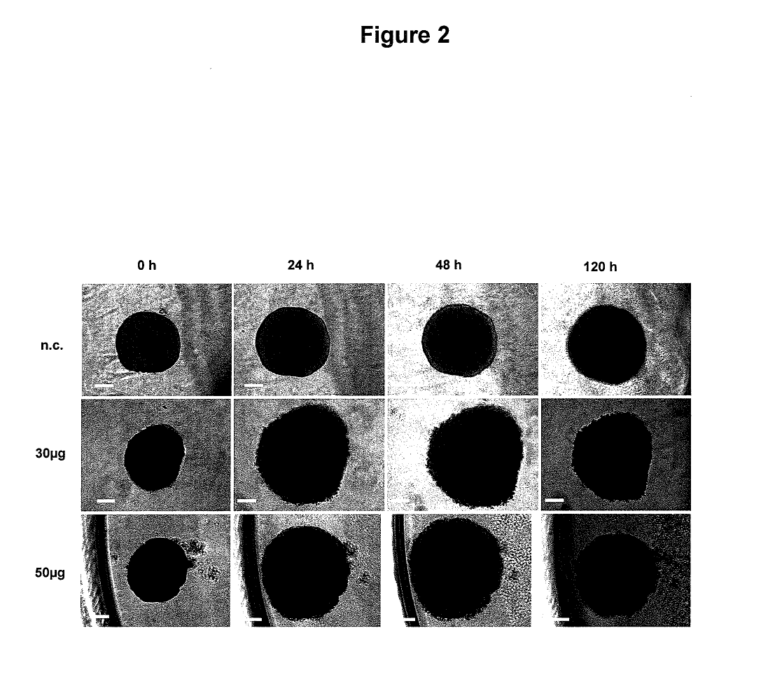

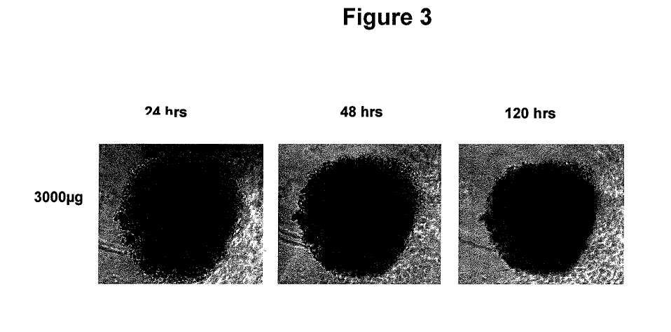

A blank, i.e. melanoma cells without any cytotoxic agent, and an equal amount of melanoma cells together with 30, 50 or 3,000 μg/ml of 3-O-acetyl altersolanol M were incubated in the matrix gel for 120 hours at 37° C. After 0, 24, 48, and 120 hours phase contrast images were taken of the samples (shown in FIGS. 2 and 3).

In the top line, FIG. 2 shows a blank (“n. c.”), which clearly shows the formation of an outer membrane and the subsequent inclusion of melanin in the condensed tumor, said tumor being constantly growing. At all three concentrations of 3-O-acetyl altersolanol M, the tumor shows signs of degeneration already after 24 hours and is clearly reduced in size after 120 hours, the degenerative effect of 3-O-acetyl altersolanol M being most significant at the highest concentration (being 100 times higher than the lowest concentration) shown in FIG. 3, as had been expected.

Thus, it was clearly shown that the present inventions provides novel compounds which are highly efficient against both multiply drug resistant bacterial and fungal pathogens and respiratory viruses and cancer cells and may, thus, be used as anti-infectives and as anti-cancer agents in a wide range of applications. The compound (4), 3-O-acetyl altersolanol M, particularly constitutes a promising agent for all the above-mentioned fields of application.

1. (1R,2S,3S,4R)-3-Acetoxy-1,2,4,5-tetrahydroxy-7-methoxy-2-methyl-1,2,3,4-tetrahydroanthracene-9,10-dione (3-O-acetyl altersolanol M):

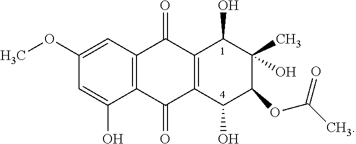

2. 8-(4,5,6-Trihydroxy-7-methyl-2-methoxy-9,10-dioxo-9H,10H-anthracen-1-yl)-(1S,2S,3R,4S)-1,2,3,4,5-pentahydroxy-7-methoxy-2-methyl-1,2,3,4-tetrahydro-9H,10H-anthracene-9,10-dione (atropisomers alterporriol I and J):

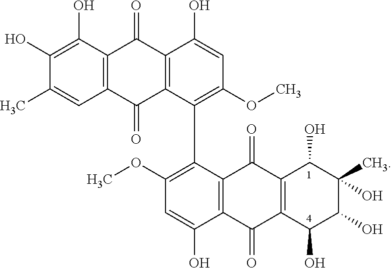

3. A use of a compound according to claim 1 or claim 2 as anti-infective.

4. The use according to claim 3, characterized in that the compound is used against Gram-positive and Gram-negative bacteria, fungi, and respiratory viruses.

5. The use according to claim 4, characterized in that the compound is used as an anti-infective against multiply drug resistant (MDR) pathogens.

6. The use according to claim 5, characterized in that the compound is used against multiply drug resistant strains of methicillin-resistant Staphylococcus aureus (MRSA), Staphylococcus epidermis, Streptococcus pneumoniae, Enterococcus faecalis or faecium, Escherichia. coli, Klebsiella sp., Pseudomonas aeruginosa, Aspergillus sp. as well as respiratory viruses from the group of human rhinoviruses and respiratory syncytial viruses.

7. A use of a compound according to claim 1 or claim 2 as an anti-cancer agent.

8. A method for producing a compound according to claim 1 or 2, characterized in that a microorganism producing the compound is fermented under growth conditions and the compound is obtained from the culture, optionally after having disrupted the cells of the microorganism.

9. The method according to claim 8, characterized in that Stemphylium globuliferum is used as the microorganism.

10. The method according to claim 8 or claim 9, characterized in that the compound is obtained by extraction and subsequent isolation from the crude extract.

11. A method for producing the compound according to claim 1, i.e. 3-O-acetyl altersolanol M, comprising the following steps:
a) syn-dihydroxylating (1R,2R)-1,2,5-trihydroxy-7-methoxy-2-methyl-1,2-dihydroanthracene-9,10-dione (6) at C3 and C4 to obtain (1R,2S,3R,4R)-3-acetoxy-1,2,4,5-tetrahydroxy-7-methoxy-2-methyl-1,2,3,4-tetrahydroanthracene-9,10-dione (7) 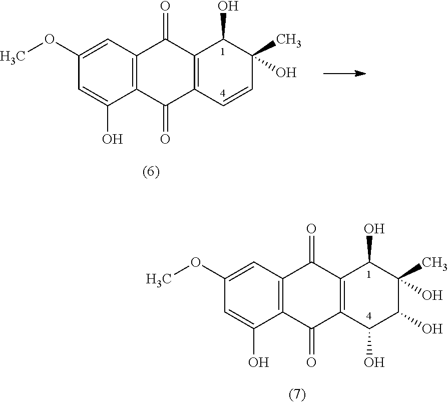
b) converting (7) into the cyclic sulfate (8) 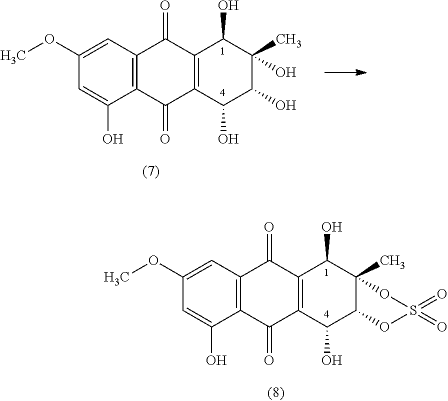
c) acidolyzing the cyclic sulfate (8) using acetic acid in order to obtain 3-O-acetyl altersolanol M.

12. A method for producing the compound according to claim 1, i.e. 3-O-acetyl altersolanol M, comprising the following steps:
a) Diels-Alder addition between naphthoquinone (9) and methylbutadiene (10), wherein the R independently represent identical or different hydroxyl protecting groups, in order to obtain tetrahydroanthraquinone (11) 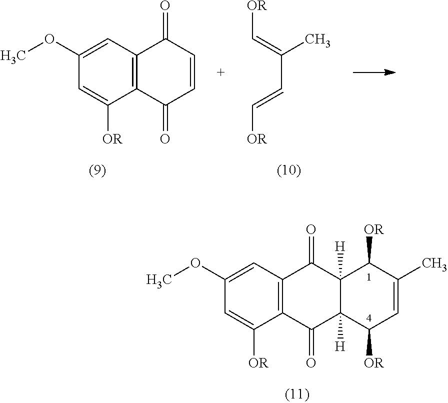
b) oxidation of the double bond between C2 and C3 using meta-chloroperbenzoic acid, carrying out a subsequent or precedent Mitsunobu reaction in order to invert the oxygen functionality at C4, in order to obtain the epoxide (12) 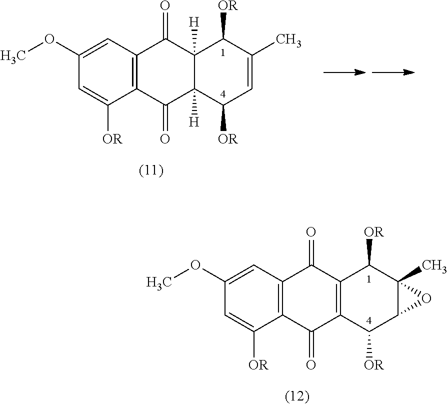
c) acidolysis of the epoxide (12) using acetic acid, subsequently or simultaneously cleaving the protecting groups R, in order to obtain 3-O-acetyl altersolanol M.

13. A method for producing the compound according to claim 1, i.e. 3-O-acetyl altersolanol M, comprising the following steps:
a) Diels-Alder addition between naphthoquinone (9) and methylbutadiene (10), wherein the R independently represent identical or different hydroxyl protecting groups, in order to obtain tetrahydroanthraquinone (11) 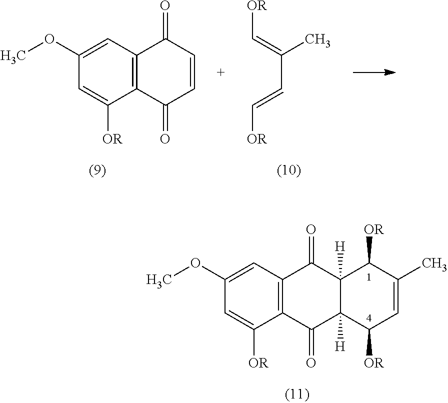
b) syn-dihydroxylation of the tetrahydroanthraquinone (11), carrying out a subsequent or precedent Mitsunobu reaction in order to invert the oxygen functionality at C4, in order to obtain the tetrahydroanthraquinone (13) 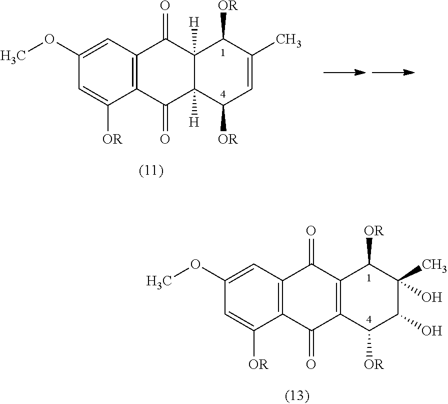
c) conversion of (13) into the cyclic sulfate (14) 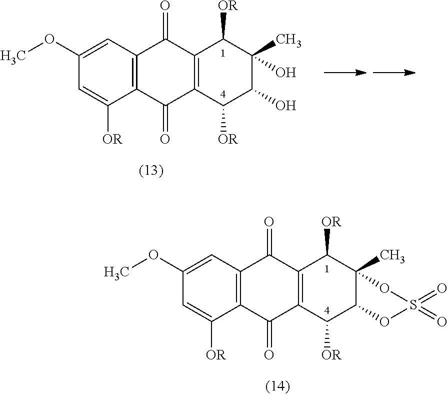
d) acidolysis of the cyclic sulfate (14) using acetic acid, subsequently or simultaneously cleaving the protecting groups R, in order to obtain 3-O-acetyl altersolanol M.
